(19) United States
(12) Patent Application Publication
(10) Pub. No.: US 2018/0110730 A1
(43) Pub. Date: Apr. 26, 2018

(54) CHEWING GUM COMPOSITION COMPRISING CANNABINOIDS AND OPIOID AGONISTS AND/OR ANTAGONISTS

(71) Applicant: AXIM Biotechnologies, Inc., New York, NY (US)

(72) Inventors: Lekhram CHANGOER, Ridderkerk (NL); George E. ANASTASSOV, New York, NY (US)

(21) Appl. No.: 15/787,498

(22) Filed: Oct. 18, 2017

Related U.S. Application Data
(60) Provisional application No. 62/410,469, filed on Oct. 20, 2016.

(51) Int. Cl. A61K 9/68; A61K 31/05; A61K 31/485; A61K 47/38; A61K 47/36; A61K 47/26; A61K 47/12; A61K 47/02; A61K 31/352

(52) U.S. Cl. CPC A61K 9/0058; A61K 31/05; A61K 31/485; A61K 47/38; A61K 31/352; A61K 47/26; A61K 47/12; A61K 47/02; A61K 47/36

(57) ABSTRACT

A chewing gum composition comprising cannabinoids or derivatives thereof and at least one opioid and optionally an opioid antagonist is provided. The chewing gum composition is formulated to provide controlled release of cannabinoids and opioid agonists and/or opioid antagonist during mastication. Methods to provide opioid addiction or dependence treatment, opioid addiction or dependence with concurrent cannabis addiction or dependence treatment, or pain treatment using the chewing gum composition according to this invention are also provided.

### CROSS-REFERENCE TO RELATED APPLICATIONS

This application claims priority to U.S. Provisional Application No. 62/410,469, filed Oct. 20, 2016, the content of which is hereby incorporated by reference in its entirety.

### BACKGROUND OF THE INVENTION

### Field of the Invention

Opioid addiction treatment and cannabis dependence treatment are developing areas with many proposed techniques and remedies. This invention involves a chewing gum composition with controlled release of cannabinoids and opioid agonists and/or antagonists for opioid and cannabis addiction and/or dependence treatment. This chewing gum may also be used for treatment of chronic pain.

### Description of the Related Technology

Opioids are a group of analgesic agents commonly used in clinical practice but are also commonly seen as addictive agents. Opioids bind to opioid receptors, which are found in the central and peripheral nervous system and the gastrointestinal tract. These receptors mediate both psychoactive and the somatic effects of opioids.

Opioids agonists include morphine, codeine, thebaine, hydrocodone, hydromorphone, oxycodone, oxymorphone, buprenorphine, fentanyl, methadone, pethidine, levorphanol, tramadol, and dextropropoxyphene.

Opioids cause euphoria and thus are used illicitly. In 2011, an estimated of 4 million people in the United States used opioids recreationally and were dependent on them. Opioid dependency may start with prescription use of opioid which turns into illicit drug use.

Physical dependence is the physiological adaptation of the body to the presence of a substance, in this case an opioid. It is the development of withdrawal symptoms when the substance is discontinued. Withdrawal symptoms of opiates may include severe dysphoria, craving, irritability, sweating, nausea, tremor, vomiting, and myalgia.

The speed and severity of withdrawal symptoms occurrence depend on half-life of the opioid. Heroin and morphine withdrawal occur more quickly and are more severe than methadone withdrawal. The acute withdrawal phase is often followed by a protracted phase of depression and insomnia that can last for months.

Addiction is marked by a change in behavior caused by the biochemical changes in the brain after continued substance abuse. Substance use becomes the main priority of the addict, regardless of the harm they may cause to themselves or others. An addiction causes people to act irrationally when they don't have the substance they are addicted to in their system. Addiction encompasses both mental and physical reliance on the substance.

Opioid addiction therapy depends on a variety of techniques. Among them is replacement therapy, wherein an opioid is replaced with another less addictive opioid, which curbs the craving feeling and reduces withdrawal symptoms, while maintains the subject's mental state such that the subject is still able to function normally.

Opioid replacement therapy is not without adverse effect. Methadone used long term may lead to potentially deadly slowed breathing. The analgesic effect in methadone ends long before the methadone half life, thus more methadone is needed, causing a built up of methadone.

The cannabis plant has many naturally occurring substances that are of great interest in the fields of science and medicine. Isolated compounds from the cannabis plant include Δ⁹-tetrahydrocannabinol (THC), cannabidiol (CBD), cannabichromene (CBC), cannabigerol (CBG), cannabidivarin (CBDV), among other compounds. While THC has psychoactive effects, CBD, CBC, CBG, and CBDV do not. Isolated compounds from the cannabis plant are called cannabinoids. There are a total of one hundred and forty one (141) cannabinoids that have been isolated from the cannabis plant. Many researchers have confirmed the medicinal value of cannabinoids. Cannabinoids have been investigated for possible treatment of seizures, nausea, vomiting, lack of appetite, pain, arthritis, inflammation, and other conditions.

The IUPAC nomenclature of THC is (-)-(6aR,10aR)-6,6,9-trimethyl-3-pentyl-6a, 7,8,10a-tetrahydro-6H-benzo[c]chromen-1-ol. CBD's IUPAC nomenclature is 2-((1S, 65)-3-methyl-6-(prop-1-en-2-yl)cyclo-hex-2-enyl)-5-pentylbenzene-1,3 -diol). CBC has the IUPAC nomenclature of 2-methyl-2-(4-methylpent-3-enyl)-7pentyl-5-chromenol. These are among the most prominent compounds in the family of compounds extracted from the cannabis plant referred to as cannabinoids.

Cannabinoids may be isolated by extraction or cold pressing of cannabis plants. Plants in the cannabis genus include Cannabis sativa, Cannabis ruderalis, and Cannabis indica. These plants are the natural sources of cannabinoids. Cannabinoids are also available in synthetic forms. Methods to synthesize cannabinoids in lab settings were discovered and are still currently practiced. Synthetic cannabinoids are more targeted, in that the synthetic compound usually comes isolated without other cannabinoids mixed in.

Cannabinoids from industrial hemp are marketed in the United States, such as cannabidiol. Various products containing cannabinoids have been marketed in recent years. Cannabinoids may be consumed by ingestion, by inhalation, via transmucosal, or by transdermal delivery.

Cannabis dependence is mainly due to Δ⁹-THC presence in cannabis. When a mammal consumes cannabis, Δ⁹-THC gives the “high” feeling. Cannabis addiction in the form of smoking cannabis also gives rise to lung cancer risk similar to tobacco smoking.

Opioid addiction treatment remains a challenge. This invention proposes a product to treat opioid addiction standing alone or concurrent opioid and Δ⁹-THC addiction/dependence.

### ABBREVIATIONS CBC: cannabichromene CBD: cannabidiol CBDV: cannabidivarin CBG: cannabigerol Δ⁹-THC: delta-9-tetrahydrocannabinol

### SUMMARY

The present invention relates to a chewing gum composition comprising cannabinoids or derivatives thereof and at least one opioid agonist and/or antagonist. Cannabinoids or derivatives thereof and opioid agonists and/or antagonists are under controlled release during mastication and consumption. This invention further relates to the use of this chewing gum composition in treating opioid addiction or dependence, concurrent cannabis and opioid addiction and/or dependence, or pain.

This invention provides a chewing composition comprising, based on total weight of the composition:

0.1 to 5% by weight of at least one cannabinoid;

0.01 to 1% by weight of at least one opioid;

20 to 95% by weight of a gum base;

5 to 35% by weight of at least one buffering agent selected from the group consisting of acetates, glycinates, phosphates, carbonates, glycerophosphates, citrates, and borates;

1 to 10% by weight of at least one flavoring agent selected from the group consisting of peppermint, spearmint, licorice, cinnamon, watermelon, vanilla, pineapple, apple, and cranberry;

1 to 65% by weight of at least one sweetening agent selected from the group consisting of isomalt, sorbitol, stevia, maltitol, and xylitol;

at least one anti-oxidant selected from the group consisting of ascorbyl palminate and sodium ascorbate; and

at least one preservative.

This invention provides a chewing gum composition according to embodiments wherein the at least one cannabinoid is cannabidiol, A⁹-tetrahydrocannabinol, cannabichromene, cannabigerol, cannabidivarin, derivatives thereof, or their acid metabolites.

This invention provides a chewing gum composition according to embodiments wherein the at least one cannabinoid is provided in combination with at least one suitable carrier selected from the group consisting of sugar alcohol, microcrystalline cellulose derivatives, dextran, agarose, agar, pectin, alginate, xanthan, chitosan, and starch.

This invention provides a chewing gum composition according to embodiments wherein the at least one cannabinoid is provided in microencapsulated form, nanoencapsulated form, or in freeze dried form.

This invention provides a chewing gum composition according to embodiments wherein the at least one cannabinoid is provided in internal voids within a suitable solid carrier.

This invention provides a chewing gum composition according to embodiments wherein the at least one cannabinoid is provided in a granule within the gum matrix.

This invention provides a chewing gum composition according to embodiments wherein the at least one cannabinoid is procured from natural sources, such as deriving or extracting from cannabis plants, or synthetic.

This invention provides a chewing gum composition according to embodiments wherein the at least one opioid is an opioid agonist selected from the group consisting of morphine, codeine, thebaine, hydrocodone, hydromorphone, oxycodone, oxymorphone, buprenorphine, fentanyl, methadone, pethidine, levorphanol, tramadol, and dextropropoxyphene.

This invention provides a chewing gum composition according to embodiments wherein the at least one opioid is an opioid prodrug selected from the group consisting of 6-monoacetyl morphine, nicomorphine, dipropanoylmorphine, desomorphine, methyldesorphine, acetylpropionyl morphine, dibenzoyl morphine, and diacetyl dihydromorphine.

This invention provides a chewing gum composition according to embodiments wherein the at least one opioid is an opioid antagonist selected from the group consisting of Naloxone hydrochloride dehydrate and natrexone hydrochloride.

This invention provides a chewing gum composition according to embodiments wherein the at least one opioid comprises both opioid agonist and opioid antagonist.

This invention provides a chewing gum composition according to embodiments wherein the at least one opioid is provided in nanoencapsulated or microencapsulated form.

This invention provides a chewing gum composition according to embodiments wherein the preservative is citric acid.

The invention provides a chewing gum composition according to embodiments which may further comprise at least one pharmaceutically acceptable excipient selected from the group consisting of fillers, disintegrants, binders, and lubricants.

This invention provides a chewing gum composition according to embodiments which may further comprise silicon dioxide or magnesium stearate.

This invention provides a method to treat opioid addiction in a mammal in need thereof, comprising administering to the mammal a chewing gum composition according to this invention.

This invention provides a method to treat opioid addiction or dependence in a mammal in need thereof, comprising administering to the mammal a chewing gum composition according to this invention, wherein the mammal receives the chewing gum administration 1 to 6 times a day.

This invention provides a method to treat opioid addiction or dependence concurrent with cannabis addiction or dependence in a mammal in need thereof, comprising administering to the mammal a chewing gum composition according to this invention.

This invention provides a method to treat opioid addiction concurrent with cannabis dependence in a mammal in need thereof, comprising administering to the mammal a chewing gum composition according to this invention, wherein the mammal receives the chewing gum administration 1 to 6 times a day.

This invention provides a method to treat pain in a mammal in need thereof, comprising administering to the mammal a chewing gum composition according to this invention.

### DETAILED DESCRIPTION OF CERTAIN INVENTIVE EMBODIMENTS

Embodiments of this application relate to a chewing gum composition comprising cannabinoids and opioid agonists and/or antagonists, wherein cannabinoids and opioid agonists and/or antagonists are incorporated into the chewing gum for controlled release. The chewing gum composition may be consumed by a human for cessation of opioid and/or cannabis dependence and/or addiction.

In embodiments, the chewing composition may comprise 0.1-5% by weight of at least one cannabinoid or derivatives thereof based on total weight of the composition. In a 2 g chewing gum piece, cannabinoids or derivatives thereof may comprise 2-100 mg. Cannabinoids in the chewing gum composition according to embodiments may be synthetic or procured from natural source.

Cannabinoids may also be in oily form, as cannabis oil, hemp oil, or hashish oil. In these embodiments, cannabinoids may be provided in a carrier to prevent absorption into the gum matrix. It is contemplated that cannabinoids provided in oily form may be used in the embodiments described herein.

Cannabinoids may be provided in a solid material carrier composed of an edible solid, such as a sugar alcohol, and cannabinoids, to prevent binding with the gum base. Cannabinoids may be embedded into the sugar alcohol. Other solids suitable for embedding cannabinoids are contemplated, such that cannabinoids are provided within internal voids of solid materials. Alternatively, cannabinoids or derivatives thereof may be provided in a granule embedded into the gum matrix. Cannabinoids provided in these manners may improve cannabinoid release during mastication of the chewing gum according to embodiments.

Cannabinoids may also be provided in microencapsulated or nanoencapsulated form or in freeze dried form. Microencapsulated or nanoencapsulated and freeze dried cannabinoids may improve the chewing gum's taste, improve stability, prevent binding with the gum matrix, control cannabinoid release during mastication, and further improve bioavailability of the cannabinoids once entering the gastrointestinal tract.

In embodiments, cannabinoids provided in encapsulated form may be particles of size 20-40 nm, which may improve bioavailability profiles of cannabinoids and prevent degradation in gastric fluid. Encapsulation may result in liposomal particles containing cannabinoids and derivatives thereof.

In these embodiments, cannabinoids may be Δ⁹-tetrahydrocannabinol (THC), cannabichromene (CBC), cannabigerol (CBG), cannabidivarin (CBDV), cannabidiol (CBD), other cannabinoids, derivatives thereof, their acid metabolites, or a combination of cannabinoids and/or their acid metabolites and/or derivatives thereof. Cannabinoids described herein may be natural or synthetic cannabinoids.

Other suitable carriers which may be combined with cannabinoids before inclusion into the gum matrix may include certain celluloses, such as microcrystalline cellulose derivatives, dextran, agarose, agar, pectin, alginate, xanthan, chitosan, or starch. The combination of cannabinoids and suitable carriers may result in cannabinoid being present within internal voids of these carriers.

Providing cannabinoids by combining with a suitable carrier or by providing cannabinoids in a capsule within the gum matrix may enable controlled release of cannabinoids during chewing of the chewing gum composition. Providing cannabinoids in microencapsulated, nanoencapsulated, or freeze dried form may also enable controlled release of cannabinoids during chewing of the chewing gum composition.

In embodiments, the chewing gum composition may further comprise at least one opioid to act as opioid agonist and/or antagonist. Opioids may be present in the chewing gum composition at 0.01-1% by weight based on the total weight composition. In a 2 g chewing gum piece, opioid may comprise 0.2-20 mg.

Opioids in the chewing gum composition according to embodiments may be opioid agonists. Opioid agonists may be derived from plant, such as morphine, codeine, or thebaine. Semi-synthetic opioids may be used, such as hydrocodone, hydromorphone, oxycodone, oxymorphone, or buprenorphine. Buprenorphine, in particular, is both an opioid agonist and antagonist, and thus its effect in opioid addiction in treatment is both as a replacement opioid and an antagonist of opioids. Synthetic opioids may also be used, such as fentanyl, methadone, pethidine, levorphanol, tramadol, or dextropropoxyphene. Prodrugs of the above opioids may also be used, such as 6-monoacetyl morphine, nicomorphine (morphine dinicotinate), dipropanoylmorphine (morphine dipropionate), desomorphine (di-hydro-desoxy morphine), methyldesorphine, acetylpropionyl morphine, dibenzoyl morphine, or diacetyl dihydromorphine.

Opioid antagonists such as Naloxone hydrochloride dehydrate (C₁₉H₂₁NO₄) and naltrexone hydrochloride (C₂₀H₂₃NO₄) may also be included in the chewing gum composition according to embodiments in addition to opioid agonists. Where opioid antagonists are included, opioid antagonists may be at 3:1 to 5:1, more specifically 4:1 ratio by weight for opioid agonist:opioid antagonist.

Certain opioids may reduce craving sensation while minimizing adverse effects on users. In opioid addiction treatments, withdrawal symptoms may be the main obstacle to recovery. When opioid agonists are provided, they may bind to opioid receptors, reducing the adverse effect of opioid withdrawal. Moreover, opioids consumed by injection, such as heroin injection, may pose additional risks to users. By providing replacement opioids in a controlled release chewing gum, replacement opioids may curb craving sensation while reducing adverse withdrawal symptoms and preventing adverse effects caused by injection. On the other hand, adding an opioid antagonist may counteract the effects of the agonists, for example the addictive opioid such as heroin, and thus eventually reduce opioid addiction.

Cannabinoids, on the other hand, may also curb craving sensation. Finally, by providing replacement opioids such as opioid agonists and/or antagonists and cannabinoids in a chewing gum form, users may avoid adverse effects caused by injection and/or smoking.

In embodiments, opioids provided may be microencapsulated or nanoencapsulated. Encapsulation may improve the chewing gum's taste as a whole. Encapsulation may aid with dissolution in the subject's oral cavity and transmucosal delivery mechanism. Encapsulation may also enable controlled release of opioids during chewing of the chewing gum composition. Encapsulation may improve opioids' bioavailability profile upon mastication of the chewing gum composition.

Methods to encapsulate opioids may be methods commonly used in the art, such as precision particle encapsulation. A method to encapsulate active ingredients in described in Berkland, C., M. King, et al. (2002). “Precise control of PLG microsphere size provides enhanced control of drug release rate.” J Control Release 82(1): 137-147. This reference is hereby included in its entirety.

Gum base provided for the chewing gun composition according to these embodiments may be non-disintegrating. Gum base such as Gum powder PG 11 TA, Gum powder PG 11 TA New, Gum powder PG 5 TA, Gum powder PG 5 TA New, and Gum powder PG N12 TA may be used. Gum base may comprise 20-95% by weight of the composition.

In embodiments, at least one buffering agent may be included in this chewing gum composition. Suitable buffering agents may include acetates, glycinates, phosphates, carbonates, glycerophosphates, citrates, borates, and/or mixtures thereof. Buffering agents may be present at 5-35% by weight.

In embodiments, the chewing gum composition may have other ingredients to improve organoleptic properties. The chewing gum composition according to embodiments may include at least one flavoring agent and at least one sweetening agent.

Ingredients such as certain flavoring agents may be included. Flavoring agents may include peppermint, spearmint, licorice, cinnamon, watermelon, vanilla, pineapple, apple, cranberry, and/or other suitable flavoring agents. Certain food colorants may be included to improve the aesthetic appearance of the chewing gum composition. Flavoring agents may be present in this chewing composition at 1-10% by weight.

Sweetening agents may be present at 1-65% of by weight of the composition according to embodiments. Sweetening agents used in chewing gums according to embodiments may be isomalt, sorbitol, stevia, maltitol, xylitol, or other suitable sweetening agents, and/or combinations thereof.

In embodiments, the chewing gum composition may comprise ingredients for preservation such as citric acid. Additional ingredient to assist with powder flow and prevent the gum base from sticking to manufacturing surfaces may be included. Such ingredient may be silicon dioxide or magnesium stearate. Other ingredients for preservation and manufacturing management may also be used.

Additional pharmaceutically acceptable excipients used in the chewing gum composition according to embodiments may be fillers, disintegrants, binders, or lubricants. Anti-oxidants such as ascorbyl palminate and sodium ascorbate may also be included. The chewing gum composition according to embodiments may comprise at least one pharmaceutically acceptable excipient and/or at least one anti-oxidant.

The chewing gum composition according to embodiments may be made by a compressing process or by a hot process. In the compressing process, ingredients are mixed and compressed into the gum base using a compress machine. In the hot process, ingredients are mixed and heated before the gum base is poured in. The gum mixture is then molded and left to cure.

The gum mixture may then be cut into appropriate size for consumption, such as 2 grams for each piece. The gum pieces may be coated with a polyol coating such as sorbitol, maltitol, isomalt, or starch. The coating layer may prevent moisture from penetrating into the gum matrix. The gum pieces may be wrapped in separate pieces of wrapping materials and packaged into a pack or box, or packaged into a blister package.

The chewing gum composition according to embodiments may be used for opioid dependence and/or addiction treatment. Opioid dependence and/or addiction may be treated or alleviated by consumption of chewing gums according to embodiments. Cannabinoids and opioid agonists and/antagonists in these chewing gums may be released in a controlled manner and absorbed by a subject via transmucosal delivery mechanism. A mammal, such as a human being, may chew the chewing gum composition according to embodiments 1-6 times a day to aid with opioid craving sensation whiling curbing this craving.

The chewing gum composition according to embodiments may be used in treatment of cannabis dependence, in particular Δ⁹-tetrahydrocannabinol dependence, concurrent with opioid addiction. Cannabis dependence, in particular Δ⁹-THC dependence, concurrent with opioid addiction may be treated or alleviated by consumption of chewing gums according to embodiments. A mammal, such as a human being, may chew the chewing gum composition according to embodiments 1-6 times a day to aid opioid and/or Δ⁹-THC craving sensation while curbing this craving.

The chewing gum composition according to embodiments may be used in treatment of pain and/or chronic pain. A mammal, such as a human being, may chew the chewing gum composition according to embodiments as need for treatment of pain. A mammal, such as a human being, may chew the chewing gum composition according to embodiments 1-6 times a day to treat and/or alleviate pain.

### EXAMPLES

### Example 1

Chewing Gum Composition Preparation

Chewing gum compositions having 10 mg of CBD and 1 mg of buprenorphine are prepared by cold pressing. Percentages are given in weight percentage.

TABLE 1

| Phase | Raw material | Percentage (%) |
|---|---|---|
| A1 | Isomalt | 28.89 |
| A2 | CBD (microencapsulated) | 0.50 |
| A3 | Cellulose | 0.50 |
| A4 | Buprenorphine (microencapsulated) | 0.04 |
| A5 | Naltrexone hydrochloride (microencapsulated) | 0.01 |
| B1 | Gum base | 24.50 |
| B2 | Sorbitol | 10.00 |
| B3 | Maltitol | 10.00 |
| B4 | Citric acid | 0.50 |
| B5 | Magnesium stearate | 2.00 |
| B6 | Silicon dioxide | 0.40 |
| B7 | Xylitol | 13.60 |
| B8 | Stevia | 1.05 |
| B9 | Vanilla | 4.00 |
| B10 | Spearmint/peppermint | 4.00 |
| B11 | Colorants FD&C blue | 0.01 |
| | Total | 100.00 |

Step 1: Make a blend of A2, A3, A4, and A5 into A1 to form Phase 1.

Step 2: Mix B1-B11 in a separate vessel to form Phase 2.

Step 3: Use a double layer chewing gum machine to compress Phase 1 and Phase 2 together.

Chewing gum composition may be cut into 2 gram pieces as appropriate. Chewing gum compositions with a mass at about 2 grams and containing 10 mg of CBD and 1 mg of buprenorphine were prepared.

### Example 2

Chewing Gum Composition Preparation

Chewing gum compositions having 10 mg of Δ⁹-THC and 1 mg of buprenorphine are prepared. Percentages are given in weight percentage.

TABLE 2

| Phase | Raw material | Percentage (%) |
|---|---|---|
| A1 | Gum base | 75.00 |
| A2 | Xylitol | 14.00 |
| A3 | Glycerine | 4.50 |
| B1 | Sacharrine | 0.40 |
| B2 | Water | 1.50 |
| B3 | Buprenorphine (microencapsulated) | 0.05 |
| B4 | Citric acid | 0.50 |
| C1 | Peppermint aroma oil | 1.50 |
| A4 | Vanilla/cranberry flavor | 1.50 |
| C2 | Δ⁹-THC (microencapsulated) | 0.50 |
| A5 | Cellulose | 0.50 |
| | Total | 100.00 |

Step 1: Heat the gum base (A1) to 90° C., then add A2-A5 to form Phase 1.

Step 2: Dissolve B1 and B4 in B2 to form Phase 2.

Step 3: Heat the peppermint oil (C1) to 60-70° C., then add C2 and B3 to form Phase 3.

Step 4: Add Phase 2 to Phase 1, stir vigorously and add Phase 3, stir for 7 minutes.

Step 5: Pour the gum mixture out and prepare chewing gum tablets by molding as need.

Chewing gum composition may be cut into 2 gram pieces as appropriate. Chewing gums with a mass at about 2 grams and containing 10 mg of Δ⁹-THC and 1 mg of buprenorphine were prepared.

### Example 3

Chewing Gum Composition Preparation

Chewing gums having 10 mg of CBD, 0.8 mg of thebaine, and 0.2 mg of Naloxone hydrochloride are prepared. Percentages are given in weight percentage.

TABLE 3

| Phase | Raw material | Percentage (%) |
|---|---|---|
| A1 | Isomalt | 28.89 |
| A2 | CBD (microencapsulated) | 0.50 |
| A3 | Cellulose | 0.50 |
| A4 | Thebaine (microencapsulated) | 0.04 |
| A5 | Naloxone hydrochloride (microencapsulated) | 0.01 |
| B1 | Gum base | 24.50 |
| B2 | Sorbitol | 10.00 |
| B3 | Maltitol | 10.00 |
| B4 | Citric acid | 0.50 |
| B5 | Magnesium stearate | 2.00 |
| B6 | Silicon dioxide | 0.40 |
| B7 | Xylitol | 13.60 |
| B8 | Stevia | 1.05 |
| B9 | Licorice | 4.00 |
| B10 | Spearmint | 4.00 |
| B11 | Colorants FD&C blue | 0.01 |
| | Total | 100.00 |

Step 1: Make a blend of A2, A3, A4, and A5 into A1 to form Phase 1.

Step 2: Mix B1-B11 in a separate vessel to form Phase 2.

Step 3: Use a double layer chewing gum machine to compress Phase 1 and Phase 2 together.

Chewing gum composition may be cut into 2 gram pieces as appropriate. Chewing gums with a mass at about 2 grams and containing 10 mg of CBD, 0.8 mg of thebaine (semi-synthetic), and 0.2 mg of Naloxone hydrochloride were prepared.

Variations and modifications will occur to those of skill in the art after reviewing this disclosure. The disclosed features may be implemented, in any combination and sub-combination (including multiple dependent combinations and sub-combinations), with one or more other features described herein. The various features described or illustrated above, including any components thereof, may be combined or integrated in other systems. Moreover, certain features may be omitted or not implements.

Examples of changes, substitutions, and alterations are ascertainable by one skilled in the art and could be made without departing from the scope of the information disclosed herein. All references cited are hereby incorporated by reference herein in their entireties and made part of this application.

What is claimed is:

1. A chewing gum composition comprising, based on total weight of the composition:
0.1 to 5% by weight of at least one cannabinoid;
0.01 to 1% by weight of at least one opioid;
20 to 95% by weight of a gum base;
5 to 35% by weight of at least one buffering agent selected from the group consisting of acetates, glycinates, phosphates, carbonates, glycerophosphates, citrates, and borates;
1 to 10% by weight of at least one flavoring agent selected from the group consisting of peppermint, spearmint, licorice, cinnamon, watermelon, vanilla, pineapple, apple, and cranberry;
1 to 65% by weight of at least one sweetening agent selected from the group consisting of isomalt, sorbitol, stevia, maltitol, and xylitol;
at least one anti-oxidant selected from the group consisting of ascorbyl palminate and sodium ascorbate; and
at least one preservative.

2. The chewing gum composition of claim 1, wherein the at least one cannabinoid is cannabidiol, Δ⁹-tetrahydrocannabinol, cannabichromene, cannabigerol, cannabidivarin, derivatives thereof, or their acid metabolites.

3. The chewing gum composition of claim 1, wherein the at least one cannabinoid is provided in combination with at least one suitable carrier selected from the group consisting of sugar alcohol, microcrystalline cellulose derivatives, dextran, agarose, agar, pectin, alginate, xanthan, chitosan, and starch.

4. The chewing gum composition of claim 1, wherein the at least one cannabinoid is provided in microencapsulated form, nanoencapsulated form, or in freeze dried form.

5. The chewing gum composition of claim 1, wherein the at least one cannabinoid is provided in internal voids within a suitable solid carrier.

6. The chewing gum composition of claim 1, wherein the at least one cannabinoid is provided in a granule within the gum matrix.

7. The chewing gum composition of claim 1, wherein the at least one cannabinoid is procured from natural sources or synthetic.

8. The chewing gum composition of claim 1, wherein the at least one opioid is an opioid agonist selected from the group consisting of morphine, codeine, thebaine, hydrocodone, hydromorphone, oxycodone, oxymorphone, buprenorphine, fentanyl, methadone, pethidine, levorphanol, tramadol, and dextropropoxyphene.

9. The chewing gum composition of claim 1, wherein the at least one opioid is an opioid prodrug selected from the group consisting of 6-monoacetyl morphine, nicomorphine, dipropanoylmorphine, desomorphine, methyldesorphine, acetylpropionyl morphine, dibenzoyl morphine, and diacetyl dihydromorphine.

10. The chewing gum composition of claim 1, wherein the at least one opioid is an opioid antagonist selected from the group consisting of Naloxone hydrochloride dehydrate and natrexone hydrochloride.

11. The chewing gum composition of claim 1, wherein the at least one opioid comprises both opioid agonist and opioid antagonist.

12. The chewing gum composition of claim 1, wherein the at least one opioid is provided in nanoencapsulated or microencapsulated form.

13. The chewing gum composition of claim 1, wherein the preservative is citric acid.

14. The chewing gum composition of claim 1, further comprising at least one pharmaceutically acceptable excipient selected from the group consisting of fillers, disintegrants, binders, and lubricants.

15. The chewing gum composition of claim 14, further comprising silicon dioxide or magnesium stearate.

16. A method to treat opioid addiction or dependence in a mammal in need thereof, comprising administering to the mammal the chewing gum composition of claim 1.

17. The method of claim 16, wherein the mammal receives the chewing gum administration 1 to 6 times a day.

18. A method to treat opioid addiction or dependence concurrent with cannabis addiction or dependence in a mammal in need thereof, comprising administering to the mammal the chewing gum composition of claim 1.

19. The method of claim 18, wherein the mammal receives the chewing gum administration 1 to 6 times a day.

20. A method to treat pain in a mammal in need thereof, comprising administering to the mammal the chewing gum composition of claim 1.
